(19) United States
(12) Patent Application Publication
(10) Pub. No.: US 2023/0018077 A1
(43) Pub. Date: Jan. 19, 2023

(54) MEDICAL INFORMATION PROCESSING SYSTEM, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Takuya SAKAGUCHI, Utsunomiya (JP); Yasuko FUJISAWA, Nasushiobara (JP)

(73) Assignee: CANON MEDICAL SYSTEMS CORPORATION, Otawara-shi (JP)

(21) Appl. No.: 17/811,346

(22) Filed: Jul. 8, 2022

(30) Foreign Application Priority Data
Jul. 13, 2021 (JP) 2021-115858

(51) Int. Cl. A61B 5/00

(52) U.S. Cl. CPC A61B 5/0031; A61F 2250/0002

(57) ABSTRACT

According to an embodiment, a medical information processing system includes processing circuitry. The processing circuitry is configured to acquire a first answer of a target person to a medical interview question made at a first timing and a second answer of the target person to a medical interview question made at a second timing different from the first timing, and to determine whether or not the first answer and the second answer are consistent.

### CROSS-REFERENCE TO RELATED APPLICATION

The present application claims priority based on Japanese Patent Application No. 2021-115858 filed on Jul. 13, 2021, the contents of which is incorporated herein by reference.

### FIELD

Embodiments disclosed in the present specification and the drawings relate to a medical information processing system, a medical information processing method, and a storage medium.

### BACKGROUND

Although an answer of a patient to a medical interview question is important information for estimating a state of the patient, the information is unstable information that often changes with the patient's own sensibility and feelings. At present, a medical staff member accounts for and responds to such changes and uses the changes for medical treatment. On the other hand, the automation of medical treatment using artificial intelligence (AI) is being studied. However, such changes (instability) in patients' answers to medical interview questions may have an unfavorable influence on medical treatment using AI.

### BRIEF DESCRIPTION OF THE DRAWINGS

FIG. 1 is a diagram showing an example of a configuration of a medical information processing system according to a first embodiment.

FIG. 2 is a diagram showing an example of a configuration of a user interface according to the first embodiment.

FIG. 3 is a diagram showing an example of a configuration of a medical information processing device according to the first embodiment.

FIG. 4 is a flowchart showing a flow of a series of processing steps of processing circuitry according to the first embodiment.

FIG. 5 is a diagram showing an example of answer data.

FIG. 6 is a diagram for describing interview expressions.

FIG. 7 is a diagram showing an example of answer data and state data.

FIG. 8 is a diagram for describing a case where an answer is inconsistent.

FIG. 9 is a diagram for describing a case where an answer is inconsistent.

FIG. 10 is a diagram for describing a method of determining medical validity on the basis of a time interval of answer data.

FIG. 11 is a diagram for describing a method of determining medical validity based on a time interval of answer data.

FIG. 12 is a diagram for describing a method of determining medical validity on the basis of position information of a medical diagnosis target person.

FIG. 13 is a diagram for describing a method of determining medical validity on the basis of position information of a medical diagnosis target person.

FIG. 14 is a diagram showing an example of a weighting coefficient decision method.

FIG. 15 is a diagram showing an example of a trained model.

FIG. 16 is a diagram showing an example of a weighting coefficient decision method.

FIG. 17 is a flowchart showing a flow of a series of processing steps of processing circuitry according to a second embodiment.

FIG. 18 is a diagram showing an example of a display screen of results of determining consistency and validity in an answer.

### DETAILED DESCRIPTION

Hereinafter, a medical information processing system, a medical information processing method, and a storage medium of embodiments will be described with reference to the drawings.

According to an embodiment, a medical information processing system includes processing circuitry. The processing circuitry is configured to acquire a first answer of a target person to a medical interview question made at a first timing and a second answer of the target person to a medical interview question made at a second timing different from the first timing, and to determine whether or not the first answer and the second answer are consistent. Thereby, it is possible to reduce an influence of a change in an answer when medical treatment is performed on a patient on the basis of an answer of the patient to a medical interview question.

### First Embodiment

[Configuration of Medical Information Processing System]

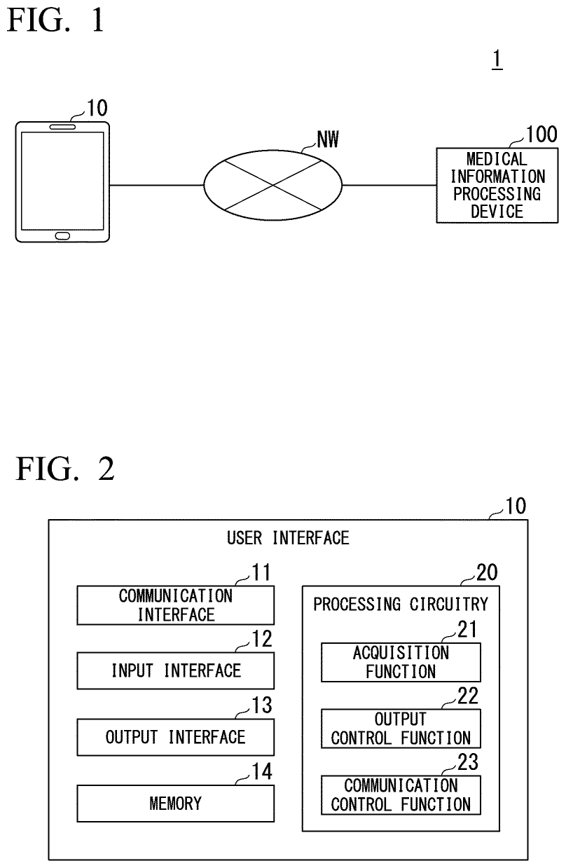

FIG. 1 is a diagram showing an example of a configuration of a medical information processing system 1 according to a first embodiment. The medical information processing system 1 includes, for example, a user interface 10 and a medical information processing device 100. The user interface 10 and the medical information processing device 100 are communicatively connected via a communication network NW.

The communication network NW may be a general information communication network using telecommunications technology. For example, the communication network NW includes a wireless/wired local area network (LAN) such as a hospital backbone LAN and an Internet network as well as a telephone communication circuit network, an optical fiber communication network, a cable communication network, a satellite communication network, and the like.

The user interface 10 is used by patients and medical staff members. For example, the user interface 10 is a touch interface or a voice user interface, and more specifically, a terminal device such as a personal computer, a tablet terminal, or a portable phone. The user interface 10 may serve as both a touch interface and a voice user interface. The medical staff member is typically a doctor, but may be a nurse or a person involved in the medical treatment. For example, the patient inputs his/her own answer to a medical interview question to the user interface 10 by touch or voice. Also, the medical staff member may verbally ask the patient the medical interview question, hear an answer to the medical interview question from the patient, and input a hearing result to the user interface 10.

In the present embodiment, “medical treatments” may include medical treatments and all other medical practices before or after the medical examination is reached as well as medical treatments such as surgery and medication.

The user interface 10 transmits information input by the patient or the medical staff member to the medical information processing device 100 via the communication network NW or receives the information from the medical information processing device 100.

The medical information processing device 100 receives information from the user interface 10 via the communication network NW and processes the received information. The medical information processing device 100 transmits the processed information to the user interface 10 via the communication network NW. In addition to or instead of transmitting the processed information to the user interface 10, the medical information processing device 100 may transmit the processed information to a dedicated terminal for a medical staff member installed in the hospital.

The medical information processing device 100 may be a single device or a system in which a plurality of devices connected via a communication network NW operate in cooperation with each other. That is, the medical information processing device 100 may be implemented by a plurality of computers (processors) included in a distributed computing system or a cloud computing system. Also, the medical information processing device 100 does not necessarily have to be a separate device different from the user interface 10 and may be a device integrated with the user interface 10.

[Configuration of Terminal Device]

FIG. 2 is a diagram showing an example of a configuration of the user interface 10 according to the first embodiment. The user interface 10 includes, for example, a communication interface 11, an input interface 12, an output interface 13, a memory 14, and processing circuitry 20.

The communication interface 11 communicates with the medical information processing device 100 or the like via the communication network NW. The communication interface 11 includes, for example, a network interface card (NIC), a wireless communication antenna, and the like.

The input interface 12 receives various input operations from an operator (for example, a patient), converts the received input operations into electrical signals, and outputs the electrical signals to the processing circuitry 20. For example, the input interface 12 includes a mouse, a keyboard, a trackball, a switch, a button, a joystick, a touch panel, a camera, or the like. The input interface 12 may be, for example, a user interface that receives a voice input such as a microphone. When the input interface 12 is a touch panel, the input interface 12 may also have a display function of a display 13a included in the output interface 13 to be described below.

In the present specification, the input interface 12 is not limited to an input interface having physical operation parts such as a mouse and a keyboard. For example, examples of the input interface 12 also include electrical signal processing circuitry that receives an electrical signal corresponding to an input operation from external input equipment provided separately from the device and outputs the electrical signal to control circuitry.

The output interface 13 includes, for example, the display 13a, a speaker 13b, and the like.

The display 13a displays various types of information. For example, the display 13a displays an image generated by the processing circuitry 20, a graphical user interface (GUI) for receiving various types of input operations from the operator, and the like. For example, the display 13a is a liquid crystal display (LCD), a cathode ray tube (CRT) display, an organic electro luminescence (EL) display, or the like.

The speaker 13b outputs information input from the processing circuitry 20 by voice.

The memory 14 is implemented by, for example, a semiconductor memory element such as a random access memory (RAM) or a flash memory, a hard disk, or an optical disc. These non-transient storage media may be implemented by other storage devices connected via the communication network NW such as a network attached storage (NAS) and an external storage server device. Also, the memory 14 may include a non-transient storage medium such as a read only memory (ROM) or a register.

The processing circuitry 20 includes, for example, an acquisition function 21, an output control function 22, and a communication control function 23. The processing circuitry 20 implements these functions by, for example, a hardware processor (a computer) executing a program stored in a memory 14 (storage circuitry).

The hardware processor in the processing circuitry 20 is, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). Instead of storing the program in the memory 14, the program may be configured to be embedded directly within the circuitry of the hardware processor. In this case, the hardware processor implements the functions by reading and executing the program embedded within the circuitry. The program may be pre-stored in the memory 14 or may be stored in a non-transitory storage medium such as a DVD or a CD-ROM and installed from the non-transitory storage medium to the memory 14 when the non-transitory storage medium is mounted in a drive device (not shown) of the user interface 10. The hardware processor is not limited to the configuration of a single circuit but may be configured as a single hardware processor obtained by combining a plurality of independent circuits to implement each function. Also, a plurality of components may be integrated into one hardware processor to implement each function.

In the acquisition function 21, input information is acquired via the input interface 12 or information is acquired from the medical information processing device 100 via the communication interface 11.

The output control function 22 causes the display 13a to display the information acquired by the acquisition function 21 or causes the information acquired by the acquisition function 21 to be output from the speaker 13b.

In the communication control function 23, the information input to the input interface 12 is transmitted to the medical information processing device 100 via the communication interface 11.

[Configuration of Medical Information Processing Device]

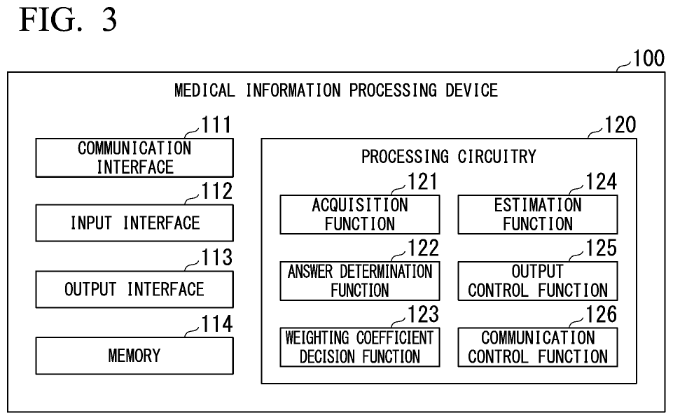

FIG. 3 is a diagram showing an example of a configuration of the medical information processing device 100 according to the first embodiment. The medical information processing device 100 includes, for example, a communication interface 111, an input interface 112, an output interface 113, a memory 114, and processing circuitry 120.

The communication interface 111 communicates with the user interface 10 and the like via the communication network NW. The communication interface 111 includes, for example, an NIC or the like.

The input interface 112 receives various types of input operations from the operator, converts the received input operations into electrical signals, and outputs the electrical signals to the processing circuitry 120. For example, the input interface 112 includes a mouse, a keyboard, a trackball, a switch, a button, a joystick, a touch panel, or the like. The input interface 112 may be, for example, a user interface that receives a voice input such as a microphone. When the input interface 112 is a touch panel, the input interface 112 may also have a display function of the display 113a included in an output interface 113 to be described below.

Also, in the present specification, the input interface 112 is not limited to an input interface having physical operation parts such as a mouse and a keyboard. For example, examples of the input interface 112 include electrical signal processing circuitry that receives an electrical signal corresponding to an input operation from external input equipment provided separately from the device and outputs the electrical signal to control circuitry.

The output interface 113 includes, for example, a display 113a, a speaker 113b, and the like. The output interface 113 is another example of the “outputter.”

The display 113a displays various types of information. For example, the display 113a displays an image generated by the processing circuitry 120, a GUI for receiving various types of input operations from the operator, and the like. For example, the display 113a is an LCD, a CRT display, an organic EL display, or the like.

The speaker 113b outputs the information input from the processing circuitry 120 by voice.

The memory 114 is implemented by, for example, a semiconductor memory element such as a RAM or a flash memory, a hard disk, or an optical disc. These non-transient storage media may be implemented by other storage devices connected via the communication network NW such as an NAS or an external storage server device. Also, the memory 114 may include a non-transient storage medium such as a ROM or a register.

The memory 114 stores a natural language processing database DB1, a medical case database DB2, and model information MI in addition to the program to be executed by the hardware processor.

The natural language processing database DB1 is a database used for natural language processing such as morphological analysis and synonym extraction.

The medical case database DB2 is a database in which a plurality of medical cases observed in the past are accumulated or organized.

The model information MI is information (a program or a data structure) that defines a certain trained model MDL1. A trained model MDL is typically a machine learning model trained to estimate a disease that the patient is already suffering from or is likely to have in the future from a patient's answer to a medical interview question. The trained model MDL may be implemented by a deep neural network(s) (DNN) such as a convolutional neural network (CNN). Also, the trained model MDL is not limited to the DNN and may be implemented by other models such as a support vector machine, a decision tree, a naive Bayes classifier, and a random forest. The details of the trained model MDL will be described below.

When the trained model MDL is implemented by the DNN, the model information MI includes, for example, concatenation information indicating how units included in each of an input layer constituting the DNN, one or more hidden layers (intermediate layers), and an output layer are concatenated to each other, weight information indicating how many concatenation coefficients are assigned to a data input/output between the concatenated units, and the like. The concatenation information is, for example, information of the number of units included in each layer, information for designating a type of unit to which each unit is concatenated, an activation function for implementing each unit, a gate provided between the units in the hidden layer, and the like. The activation function for implementing the unit may be, for example, a rectified linear unit (ReLU) function, an exponential linear units (ELU) function, a clipping function, a sigmoid function, a step function, a hyperbolic tangent function, an equality function, or the like. The gate selectively passes or weights data transmitted between the units, for example, in accordance with a value (for example, 1 or 0) returned by the activation function. The concatenation coefficient includes, for example, a weight given to the output data when data is output from a unit of a certain layer to a unit of a deeper layer in a hidden layer of a neural network. Also, the concatenation coefficient may include a bias component unique to each layer and the like.

The processing circuitry 120 includes, for example, an acquisition function 121, an answer determination function 122, a weighting coefficient decision function 123, an estimation function 124, an output control function 125, and a communication control function 126. The acquisition function 121 is an example of an “acquirer,” the answer determination function 122 is an example of an “answer determiner,” the estimation function 124 is an example of an “estimator,” and the output control function 125 is an example of a “display controller.”

The processing circuitry 120 implements these functions by, for example, a hardware processor (a computer) executing a program stored in the memory 114 (storage circuitry).

The hardware processor in the processing circuitry 120 is, for example, circuitry such as a CPU, a GPU, an ASIC, or a programmable logic device (for example, an SPLD, a CPLD, or an FPGA). Instead of storing the program in the memory 114, the program may be configured to be embedded directly within the circuitry of the hardware processor. In this case, the hardware processor implements the functions by reading and executing the program embedded within the circuitry. The program may be pre-stored in the memory 114 or may be stored in a non-transitory storage medium such as a DVD or a CD-ROM and installed from the non-transitory storage medium to the memory 114 when the non-transitory storage medium is mounted in a drive device (not shown) of the medical information processing device 100. The hardware processor is not limited to the configuration of a single circuit but may be configured as a single hardware processor obtained by combining a plurality of independent circuits to implement each function. Also, a plurality of components may be integrated into one hardware processor to implement each function.

[Processing Flow of Medical Information Processing Device]

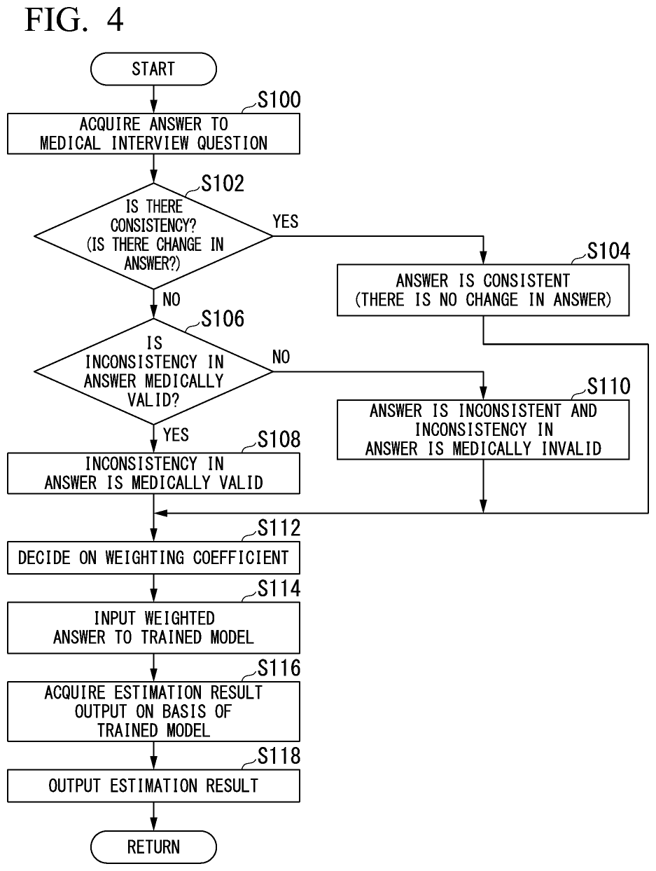

Hereinafter, a series of processing steps of the processing circuitry 120 of the medical information processing device 100 will be described with reference to the flowchart. FIG. 4 is a flowchart showing a flow of a series of processing steps of the processing circuitry 120 according to the first embodiment.

First, in the acquisition function 121, answer data of the patient to be medically diagnosed (hereinafter referred to as a medical diagnosis target person) to a medical interview question is acquired from the user interface 10 via the communication interface 111 (step S100). The answer data is qualitative digital data including the subjectivity of the medical diagnosis target person, and is, for example, text data. When the answer to the medical interview question is input by voice, voice data may be converted into text by voice recognition technology.

For example, in the acquisition function 121, answer data answered by the same medical diagnosis target person is acquired at each of a plurality of different timings. Specifically, in the acquisition function 121, answer data answered by the medical diagnosis target person X in the early morning, answer data answered by the medical diagnosis target person X in the daytime, answer data answered by the medical diagnosis target person X in the evening, and the like may be acquired. The early morning is an example of a “first timing,” the answer data answered by the medical diagnosis target person X in the early morning is an example of a “first answer,” the daytime or the evening is an example of a “second timing,” and the answer data answered by the medical diagnosis target person X in the daytime or evening is an example of a “second answer.”

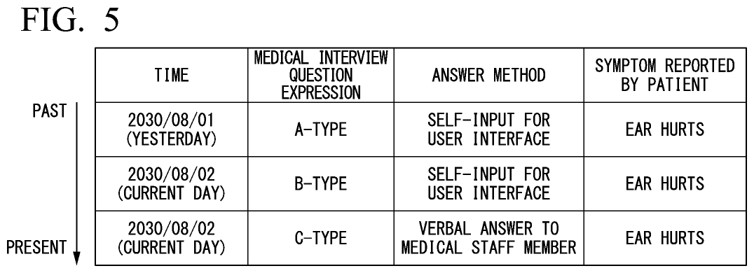

FIG. 5 is a diagram showing an example of answer data. As shown in FIG. 5, a medical interview question expression, an answer method, a symptom self-reported by the patient, and the like are associated with a time when the medical diagnosis target person has answered in the answer data. The medical interview question expression indicates an expression method for medical interview question content (subjective symptoms, worries, and the like). The answer method indicates whether the patient himself/herself has input the answer to the user interface 10 or whether the patient has verbally answered the medical staff member and the medical staff member has input the answer heard from the patient to the user interface 10. The time when the medical diagnosis target person has answered may be the time when the medical diagnosis target person has asked the medical interview question.

For example, the answer data on 2030/08/01 corresponds to an A-type medical interview question expression and indicates that the patient himself/herself input the answer to the user interface 10 and reported the symptom of pain in his/her ear. The answer data on 2030/08/02 corresponds to a C-type medical interview question expression and indicates that the patient verbally answered the medical staff member and reported the symptom of pain in his/her ear.

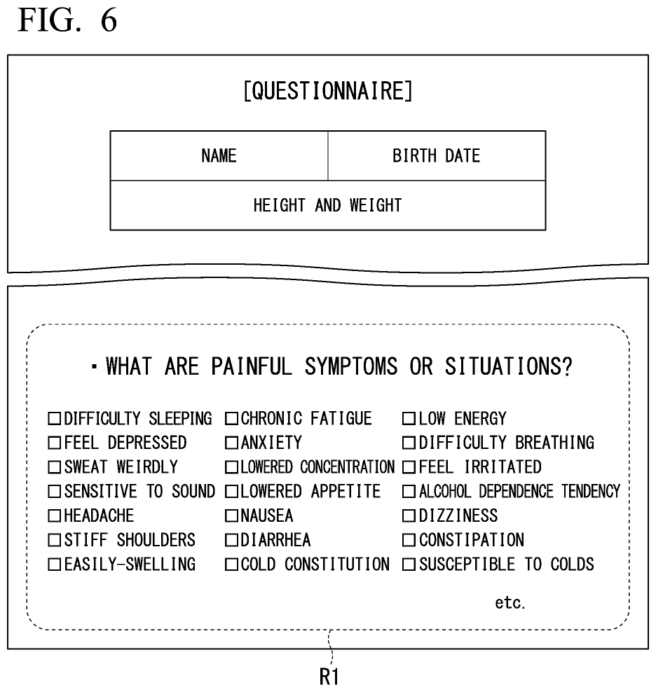

FIG. 6 is a diagram for describing the medical interview question expression. As described above, there are several types of medical treatment expressions such as an A-type, a B-type, and a C-type. For example, although content of the medical interview question at a certain timing is the same as content of the medical interview question at another timing, the expressions thereof may be different from each other. This expression difference will be described using a questionnaire shown in FIG. 6 as an example. For example, when medical treatment from a medical institution is performed for a medical diagnosis target person, he/she is required to fill necessary items (R1 in FIG. 6) such as current physical and mental states, a medical history, and the presence/absence of allergies in the questionnaire.

For example, when a medical interview question associated with the content of “sleep” is taken into account, there are several expressions such as “difficulty sleeping,” “sleep during the day,” “too sleep,” and “lack of sleep” as medical interview question expressions. Even if the medical interview questions have the same content of “sleep,” the expression thereof may differ according to a patient and therefore the answer different from the subjective symptom of the patient may be made. In other words, even in the medical interview question with respect to the same content, the content of the answer may change due to the different expressions. This indicates that the answers to the medical interview questions have changed. The answer data includes the expression of the medical interview question to determine a change in the answer due to the difference in the expression of the medical interview question.

Also, the questionnaire may be displayed on the display 13a of the user interface 10 or may be printed on paper and distributed to the patient. When the patient has filled answers in the paper questionnaire, the medical staff member of the medical institution may input written content to the user interface 10. At this time, an optical character recognition/reader (OCR) may be used. Question content of the questionnaire may be output as voice from the speaker 113b of the user interface 10 or may be verbally read by the medical staff member of the medical institution. When the patient has spoken an answer to the user interface 10, the user interface 10 may acquire the answer spoken by the patient via a microphone. Alternatively or additionally, the medical staff member may hear the answer spoken by the patient. When the medical staff member hears the answer from the patient, a hearing result may be input to the user interface 10. Also, it is not necessary to determine the questions in advance as in the questionnaire and the medical staff member may freely determine content of the medical interview questions at the timing of the medical examination. The answer to the medical interview question is medically called a chief complaint. Thus, the answer data may be read as chief complaint data.

In the acquisition function 121, state data and treatment data of the medical diagnosis target person may be further acquired from the user interface 10 and other equipment via the communication interface 111.

The state data is quantitative digital data obtained by sensing the state of the medical diagnosis target person from the appearance. For example, the state data is face image data, voice data, or the like of the medical diagnosis target person. A face of the medical diagnosis target person may be imaged by, for example, a camera of the user interface 10, or may be imaged by the camera installed in a medical examination room. Likewise, the voice of the medical diagnosis target person may be collected by, for example, the microphone of the user interface 10, or may be collected by the microphone installed in the medical examination room. Also, the state data may include objective observation results of a doctor.

The treatment data is quantitative digital data in which biological information of the medical diagnosis target person is measured by various pieces of treatment equipment. The treatment equipment is a device for medically examining a patient, for example, an X-ray computed tomography (CT) device, a magnetic resonance imaging (MRI) device, a mammography device, a sonic image diagnostic device, a nuclear medicine diagnostic device, a body fluid analysis device, a vital sign measurement device, or the like. That is, the treatment data is an MR image, a CT image, vital sign data, or the like.

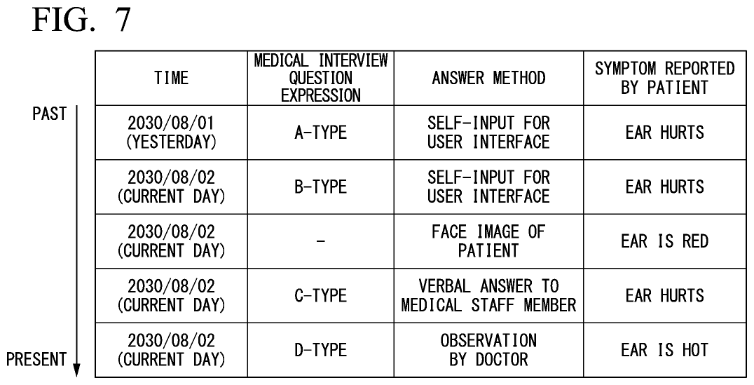

FIG. 7 is a diagram showing an example of answer data and state data. For example, the patient's face image data and the symptom estimated from the face image data may be associated with the time when the patient's face image has been captured. Also, the symptom diagnosed by the doctor from the observation result may be associated with the time when the doctor has observed the patient.

Also, in the acquisition function 121, position information (for example, coordinates measured by a global positioning system (GPS) receiver of the user interface 10) may be further acquired from the user interface 10 via the communication interface 111 or an operation quantity at the time of a touch input or an operation quantity at the time of a voice input may be further acquired.

The flowchart in FIG. 4 will be described continuously. Subsequently, in the answer determination function 122, a plurality of pieces of answer data having different timings from each other are compared and it is determined whether or not the answer data is consistent (step S102). That is, in the answer determination function 122, it is determined whether or not the answer to the medical interview question has changed.

For example, in the answer determination function 122, natural language processing may be performed on each of the plurality of pieces of answer data to be compared while referring to the natural language processing database DB1 and it may be determined whether or not the answer data is consistent. The natural language processing may include a process of converting onomatopoeia such as “tingling” or an adjective such as “red” into a word(s) for expressing the symptom such as “hurt” or “there is a fever” (a process of classifying a related language).

Also, in the answer determination function 122, it may be determined whether or not the answer data is consistent on the basis of state data of the medical diagnosis target person at each timing in addition to or in place of a process of determining whether or not the answer data is consistent according to the natural language processing.

For example, in the answer determination function 122, image processing is performed on the face image data of the medical diagnosis target person, characteristic parts such as ears, nose, and eyes are extracted from the image, and the colors of these parts are further identified. For example, in the answer determination function 122, it is assumed that an “ear” portion is extracted from the face image and it is identified that the “ear” is red. In this case, in the answer determination function 122, the determination result may be converted into a character string “ear is red” as shown in FIG. 7. In the answer determination function 122, the adjective “red” included in the character string is converted into a word for expressing a symptom such as “hurt” using the natural language processing and the character string “ear hurts” after the conversion (i.e., the character string derived from the image data) is compared with the character string included in the answer data, such that it may be determined whether or not the answer data is consistent with the image data.

Also, in the answer determination function 122, voice data is converted into a character string by performing a voice recognition process on the voice data of the medical diagnosis target person and the character string derived from the voice data is compared with the character string included in the answer data, such that it may be determined whether or not the answer data is consistent with the voice data.

In the answer determination function 122, when character strings (character strings each representing the symptom reported by the patient) included in the plurality of pieces of answer data to be compared are consistent with each other, it is determined that the answer data is consistent (the answer has not changed) (step S104). Also, in the answer determination function 122, when the character string derived from the state data such as the image data or the voice data and the character string included in the answer data are consistent with each other, it may be determined that the answer data is consistent with the state data (the answer has not changed).

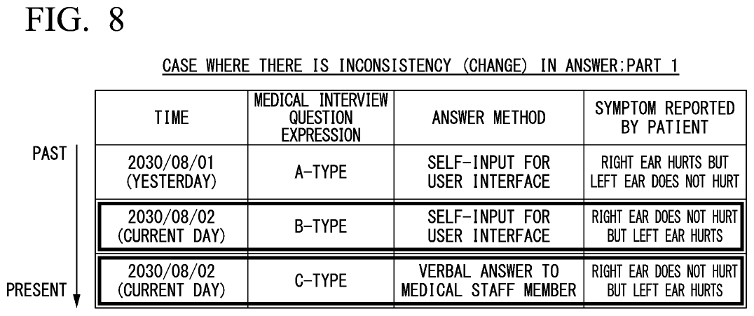

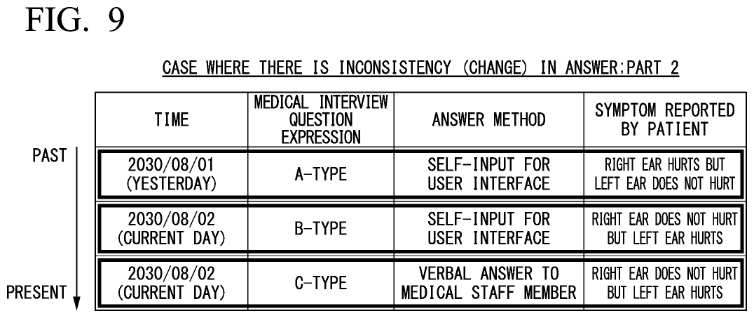

FIGS. 8 and 9 are diagrams for describing cases where the answers are inconsistent. In the example of FIG. 8, first answer data indicating a symptom of “right ear hurts,” while second and third answer data indicates a symptom of “left ear hurts.” In this case, in the answer determination function 122, it is determined that the answer data is inconsistent.

In the example of FIG. 9, the first answer data indicates the symptom of “right ear hurts,” the second answer data indicates the symptom of “left ear hurts,” and the third answer data also indicates the symptom “right ear hurts” like the first answer data. Even if the symptom is different every time the medical interview question is made in this way, it is determined that the answer data is inconsistent in the answer determination function 122.

When it is determined that the answer data is inconsistent in the answer determination function 122, the inconsistent answer data is output via the output interface 113 in the output control function 125.

For example, as shown in FIGS. 8 and 9, the output control function 125 may cause the display 113a to display three pieces of answer data in which the inconsistency has occurred in chronological order. At this time, the output control function 125 may change a display mode of each of the three pieces of answer data in which the inconsistency has occurred.

In the case of an example of FIG. 8, in the output control function 125, the first answer data is correct and the second and third answer data inconsistent with the first answer data may be highlighted as compared with the first answer data. In the case of the example of FIG. 9, all the three pieces of answer data may be highlighted and displayed in the output control function 125.

By displaying the answer data in chronological order in this way, the medical staff member can visually know from what timing the answer to the medical interview question has begun to change.

Also, in the output control function 125, the answer data consistent with each other may be grouped and the display 113a may be allowed to display the answer data side by side for each group. For example, in the output control function 125, groups may be classified into a group of answer data indicating a symptom of “right ear hurts” and a group of answer data indicating a symptom of “left ear hurts.” Thereby, the medical staff member can visually see how the answer has changed.

Also, in the communication control function 126, answer data in which inconsistency has occurred may be transmitted to the user interface 10 via the communication interface 111. In the output control function 22 of the user interface 10, when the communication interface 11 receives the inconsistent answer data from the medical information processing device 100, the answer data may be displayed as an image on the display 13a of the output interface 13 or may be output as voice from the speaker 13b.

The flowchart of FIG. 4 will be described continuously. In the answer determination function 122, when it is determined that there is inconsistency in the answer data (there is a change in the answer), it is further determined whether or not the inconsistency in the answer (the change in the answer) is medically valid using the medical case database DB2 or AI (step S106).

Even if the answer to the medical interview question is inconsistent, the inconsistency itself can be valuable information for medical treatment medically. Accordingly, in the answer determination function 122, it is determined whether or not the inconsistency in the answer (the change in the answer) is medically valid to find out an inconsistent answer that can be useful information from among answers in which the inconsistency has occurred.

Furthermore, in the answer determination function 122, it may be determined whether or not the inconsistency is medically valid on the basis of a time interval of the answer data in which the inconsistency has occurred.

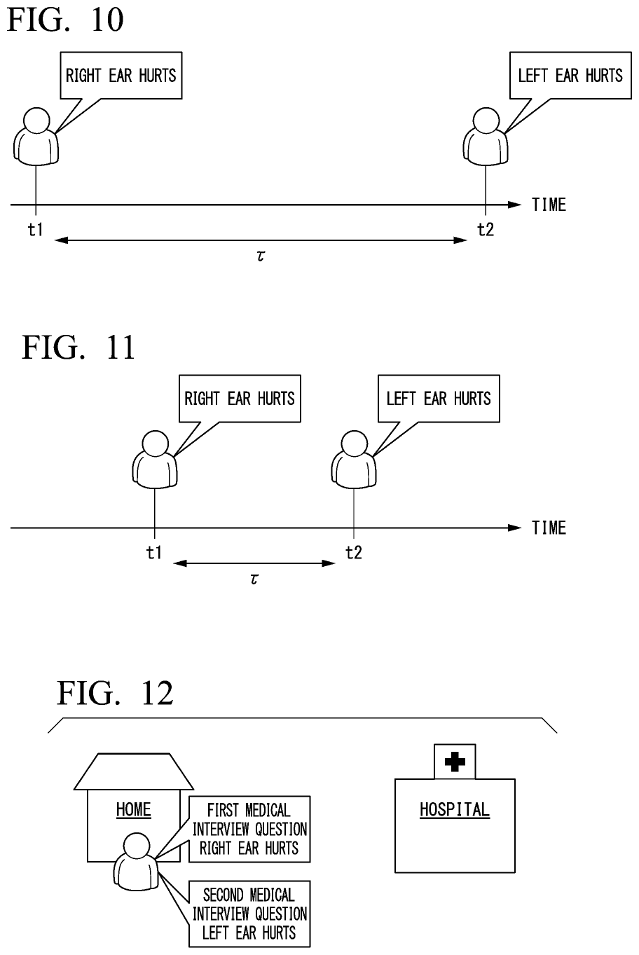

FIGS. 10 and 11 are diagrams for describing a method of determining medical validity on the basis of a time interval of answer data. For example, it is assumed that the medical diagnosis target person makes an answer of “right ear hurts” to the medical interview question at time t1 and then the medical diagnosis target person makes an answer of “left ear hurts” to the medical interview question at time t2. In this case, in the answer determination function 122, it may be determined that the inconsistency is more medically valid as an interval r from time t1 to time t2 increases and determine that the inconsistency is more medically invalid as the interval r decreases. For example, when an answer at a certain time is inconsistent with an answer one minute later, the answer has changed in just one minute. When the inconsistency has occurred at such a short interval, it may be determined that the inconsistency is medically invalid in the answer determination function 122. Also, for example, when an answer at a certain time is inconsistent with an answer one day later, it may be determined that the inconsistency is medically valid in the answer determination function 122 because it is possible to estimate that the answer has changed due to the progress of the disease.

Also, in the answer determination function 122, it may be determined whether or not the inconsistency in the answer data is medically valid on the basis of the position information when the medical diagnosis target person has answered the medical interview question.

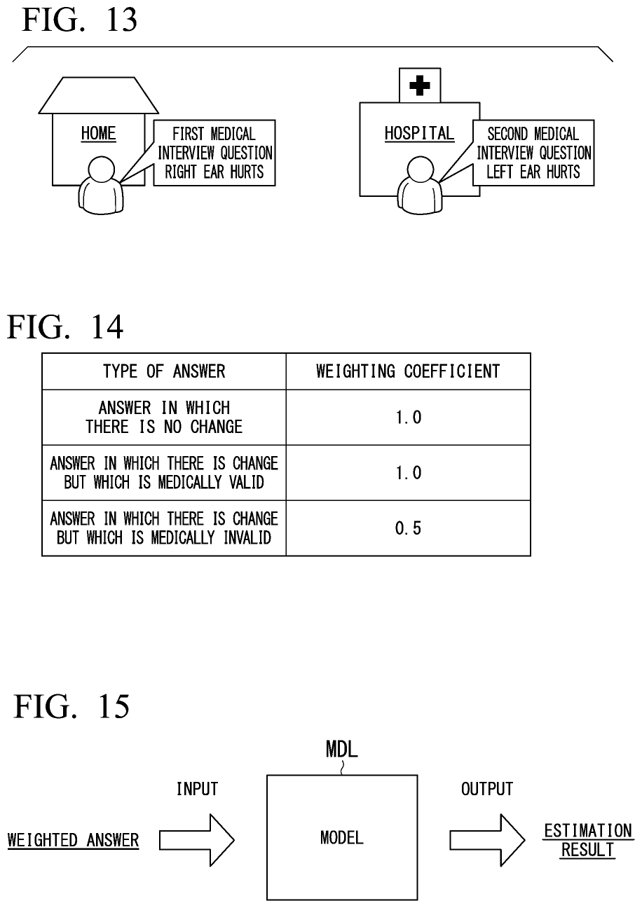

FIGS. 12 and 13 are diagrams for describing a method of determining medical validity on the basis of position information of the medical diagnosis target person. As shown in FIG. 12, for example, it is assumed that the medical diagnosis target person has answered the medical interview question twice at home using the user interface 10, and the first answer data and the second answer data are inconsistent with each other. In this case, the medical diagnosis target person is in the same place as his/her home and it can be determined that the answer has changed even though the environment at the time of answering has not changed. Thus, in the answer determination function 122, it may be determined that the inconsistency is medically valid.

As shown in FIG. 13, it is assumed that the medical diagnosis target person has answered a first medical interview question at home using the user interface 10 and then has moved to a hospital to answer a second medical interview question, and first answer data and second answer data are inconsistent with each other. In this case, it can be determined that the answer has changed due to a psychological factor or an environmental factor such as the white coat of the medical staff member. Therefore, in the answer determination function 122, it may be determined that the inconsistency is medically invalid.

Also, in the answer determination function 122, it may be determined whether or not the inconsistency in the answer data is medically valid on the basis of an operation quantity for the user interface 10 when the medical diagnosis target person has answered the medical interview question.

For example, it is assumed that the medical diagnosis target person has input the answer to the medical interview question to the user interface 10 by touch at two different timings. In this case, in the answer determination function 122, a difference or a ratio between at least one of a pressure, time, and the number of operations when the medical diagnosis target person has input a first answer to the user interface 10 by touch and at least one of a pressure, time, and the number of operations when the medical diagnosis target person has input a second answer to the user interface 10 by touch may be calculated and it may be determined whether or not inconsistency in answer data is medically valid on the basis of the difference or the ratio.

For example, because the medical diagnosis target person has pressed a touchpad or a key normally during a first answer, but has pressed the touchpad or key while shaking his/her hand during a second answer, the number of presses per unit time during the second answer is significantly larger than the number of presses per unit time during the first answer. In this case, it can be estimated that the disease has progressed from the first answer to the second answer, the hand has been shaken, and the number of presses per unit time has increased as a result. Under such estimation, the answer determination function 122 may determine that the inconsistency in the answer data is medically valid.

Likewise, for example, it is assumed that the medical diagnosis target person has pressed the touchpad or key normally during the first answer, but has strongly pressed the touchpad or key during the second answer, such that the pressure on the touchpad or key during the second answer is significantly higher than the pressure on the touchpad or key during the first answer. In this case, it can be estimated that the disease progressed from the first answer to the second answer and the pressure at the time of pressing increased as a result. Under such estimation, in the answer determination function 122, it may be determined that the inconsistency in the answer data is medically valid.

Also, the same is true for a case where the medical diagnosis target person inputs the answer to the medical diagnosis target person to the user interface 10 by voice at two different timings. For example, in the answer determination function 122, a difference or a ratio between at least one of a sound pressure, time, and the number of operations when the medical diagnosis target person has input a first answer to the user interface 10 by voice and at least one of a sound pressure, time, and the number of operations when the medical diagnosis target person has input a second answer to the user interface 10 by voice may be calculated and it may be determined whether or not inconsistency in answer data is medically valid on the basis of the difference or the ratio.

Also, in the answer determination function 122, it may be determined whether or not the inconsistency in the answer data is medically valid on the basis of image data obtained by imaging the medical diagnosis target person when the medical diagnosis target person has answered the medical interview question and voice data spoken by the medical diagnosis target person when the medical diagnosis target person has answered the medical interview question.

The flowchart of FIG. 4 will be described continuously. In the answer determination function 122, when it is determined that the inconsistency in the answer (the change in the answer) is medically valid, the answer of the medical diagnosis target person to the medical interview question is inconsistent, but the inconsistency is determined to be medically valid (step S108). On the other hand, in the answer determination function 122, when it is determined that the inconsistency in the answer (the change in the answer) is medically invalid, the answer of the medical diagnosis target person to the medical interview question is inconsistent, but the inconsistency is determined to be medically invalid (step S110).

Subsequently, in the weighting coefficient decision function 123, a degree of reliability of answer data is decided on as a weighting coefficient on the basis of a result of determining whether or not the inconsistency in the answer data is medically valid (step S112).

For example, in the weighting coefficient decision function 123, the weighting coefficient is made relatively large when the inconsistency is medically valid, as compared with a case where the inconsistency is medically invalid.

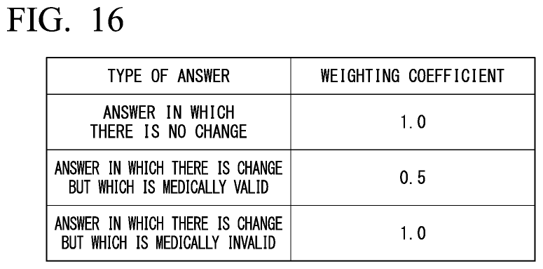

FIG. 16 is a diagram showing an example of a weighting coefficient decision method. For example, in the weighting coefficient decision function 123, the weighting coefficient may be set to 1.0 for (i) answer data in which there is no inconsistency (there is no change), the weighting coefficient may be set to 1.0 for (ii) answer data in which there is inconsistency (there is a change) but the inconsistency is medically valid, and the weighting coefficient may be set to 0.5 for (iii) answer data in which there is inconsistency (there is a change) and the inconsistency is medically invalid.

The flowchart of FIG. 4 will be described continuously. Subsequently, in the estimation function 124, answer data (i.e., weighted answer data) for which the weighting coefficient has been decided on in the weighting coefficient decision function 123 is input with respect to the trained model MDL defined by the model information MI stored in the memory 141 (step S114).

FIG. 15 is a diagram showing an example of a trained model MDL. The trained model MDL is typically a machine learning model trained using a data set in which a disease that a patient is already suffering from or is likely to have in the future is associated as a correct label (also referred to as a target) with respect to answer data of the patient of a certain learning target (hereinafter referred to as a learning target person) as training data. In other words, the trained model MDL is a machine learning model trained to output a disease that the learning target person is already suffering from or is likely to have in the future when answer data of the learning target person is input. The learning target person may be a past medical diagnosis target person. That is, the learning target person may be the same person as the medical diagnosis target person or may be a person different from the medical diagnosis target person.

As shown in FIG. 15, the trained model MDL trained using such training data will output the patient's disease as an estimation result when the answer data of a certain patient is input. A result of estimating the trained model MDL is represented by, for example, a multidimensional vector or tensor. The vector or tensor includes the likelihood (probability) of a disease as an element value. For example, it is assumed that there are a total of three types of diseases A, B, and C as diseases from which the medical diagnosis target person may suffer. In this case, the vector or tensor can be expressed as (e1, e2, e3), when the probability of disease A is e1, the probability of disease B is e2, and the probability of disease C is e3.

Also, the training data may be a data set in which a disease that the learning target person is already suffering from or is likely to have in the future is associated as a correct label (also referred to as a target) with respect to other data such as examination data or state data in addition to the answer data of the learning target person. In this case, in the estimation function 124, examination data, state data, or the like of the medical diagnosis target person is further input in addition to weighted answer data of the medical diagnosis target person with respect to the trained model MDL.

The flowchart of FIG. 4 will be described continuously. Subsequently, in the estimation function 124, a disease estimation result is acquired from the trained model MDL to which at least the weighted answer data of the medical diagnosis target person is input (step S116). The estimation result includes an estimated disease from which the medical diagnosis target person is already suffering or an estimated disease that the medical diagnosis target person is likely to have in the future.

Subsequently, in the output control function 125, a result of estimating the trained model MDL is output via the output interface 113 (step S118). Thereby, the process of the present flowchart ends.

Also, in the communication control function 126, the result of estimating the trained model MDL may be transmitted to the user interface 10 via the communication interface 111. When the communication interface 11 receives the estimation result from the medical information processing device 100, the output control function 22 of the user interface 10 may cause the display 13a of the output interface 13 to display the estimation result as an image or may cause the estimation result to be output as voice from the speaker 13b.

According to the first embodiment described above, the processing circuitry 120 of the medical information processing device 100 acquires answer data (an example of “a first answer”) of a medical diagnosis target person to a medical interview question made at a first timing and answer data (an example of “a second answer”) of the medical diagnosis target person to the medical interview question made at a second timing different from the first timing and determines whether or not the answer data is consistent. Thereby, it is possible to reduce an influence of a change in the answer when medical treatment is performed on the patient on the basis of the answer of the patient to the medical interview question.

### Second Embodiment

Hereinafter, a second embodiment will be described. The second embodiment is different from the first embodiment in that a weighting coefficient for answer data is determined in accordance with a type of disease from which a medical diagnosis target person may suffer. Hereinafter, differences from the first embodiment will be mainly described and description of parts identical to those of the first embodiment will be omitted. In the description of the second embodiment, parts identical to those of the first embodiment will be described with the same reference signs.

In the weighting coefficient decision function 123 in the second embodiment, the weighting coefficient for the answer data is decided on in accordance with the type of disease from which the medical diagnosis target person may suffer.

For example, it is known that patients with mental diseases tend to give medically and physiologically inconsistent answers. Accordingly, even if the answers of the medical diagnosis target person are inconsistent and medically invalid, it can be determined that the medical diagnosis target person is highly likely to have a mental disease if it is repeated. In such a case, as compared with (ii) answer data in which there is inconsistency (there is a change) but the inconsistency is medically valid, (iii) answer data in which there is inconsistency (there is a change) and the inconsistency is medically invalid can be more valuable information for the medical treatment of a mental disease.

Therefore, in the estimation function 124 in the second embodiment, it is estimated that there is a mental disease for the medical diagnosis target person for which (iii) answer data in which there is inconsistency (there is a change) and the inconsistency is medically invalid is repeated a given number of times or more.

In the weighting coefficient decision function 123 in the second embodiment, the weighting coefficient relatively decreases in a case where the inconsistency is medically valid as compared with a case where the inconsistency is medically invalid under a condition that the medical diagnosis target person is estimated to have a mental disease in the estimation function 124.

According to the second embodiment described above, when the medical diagnosis target person has a mental disease (or is likely to have a mental disease), the processing circuitry 120 relatively decreases the weighting coefficient in a case where the inconsistency is medically valid as compared with a case where the inconsistency is medically invalid. Thereby, the accuracy of disease estimation based on the trained model MDL can be further improved.

### Third Embodiment

Hereinafter, a third embodiment will be described. The third embodiment is different from the above-described embodiment in that a medical staff member can correct a determination result regarding the consistency and validity in an answer. Hereinafter, differences between the first embodiment and the second embodiment will be mainly described and parts identical to those of the first embodiment and the second embodiment will be omitted. In the description of the third embodiment, parts identical to those of the first embodiment or the second embodiment will be described with the same reference signs.

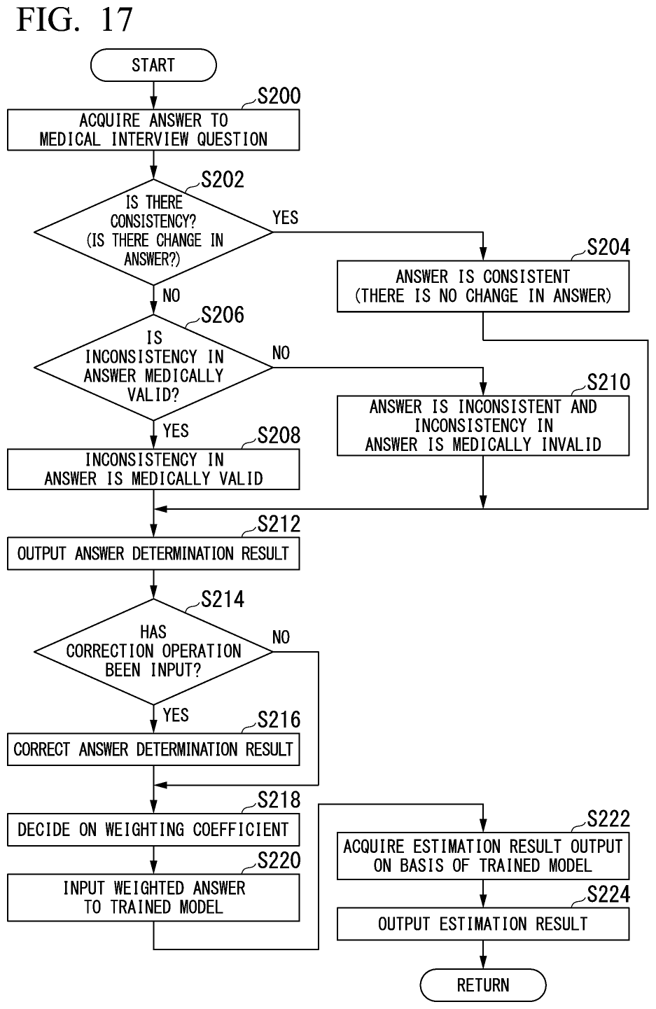

FIG. 17 is a flowchart showing a flow of a series of processing steps of processing circuitry 120 according to the second embodiment.

First, in an acquisition function 121, answer data of the medical diagnosis target person to a medical interview question is acquired from the user interface 10 via the communication interface 111 (step S200).

Subsequently, in an answer determination function 122, a plurality of pieces of answer data having timings different from each other are compared and it is determined whether or not the answer data is consistent (step S202).

In the answer determination function 122, when character strings (character strings representing symptoms reported by the patient) included in the plurality of pieces of answer data to be compared are consistent with each other, it is determined that the answer data is consistent (the answer has not changed) (step S204).

In the answer determination function 122, when it is determined that the answer data has not been consistent (the answer has changed), it is further determined whether or not the inconsistency in the answer (the change in the answer) is medically valid using the medical case database DB2 or AI (step S206).

In the answer determination function 122, when it is determined that the inconsistency in the answer (the change in the answer) is medically valid, the answer of the medical diagnosis target person to the medical interview question is inconsistent but the inconsistency is determined to be medically valid (step S208).

On the other hand, in the answer determination function 122, when it is determined that the inconsistency in the answer (the change in the answer) is medically invalid, the answer of the medical diagnosis target person to the medical interview question is inconsistent but the inconsistency is determined to be medically invalid (step S210).

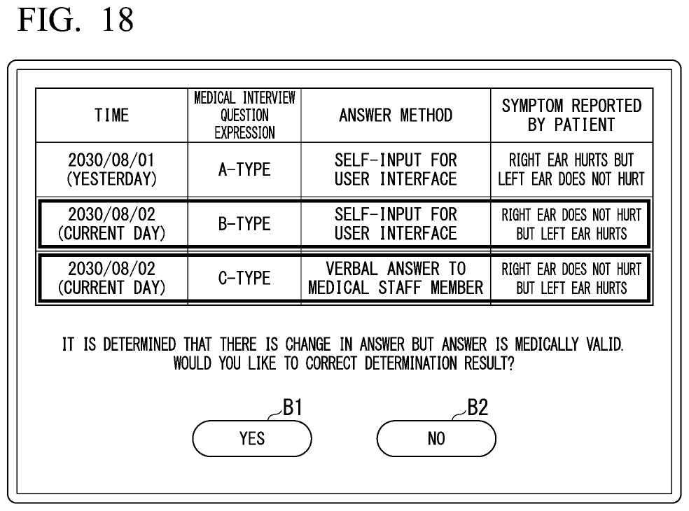

Subsequently, in an output control function 125, results of determining consistency and validity in the answer (determination results of S204, S208, and S210) are output via the output interface 113 (step S212). For example, in the output control function 125, a screen as shown in FIG. 18 to be described below may be displayed on the display 113a.

Subsequently, in the answer determination function 122, it is determined whether or not the medical staff member has input an operation of correcting the results of determining the consistency and validity in the answer (hereinafter referred to as a correction operation) to the input interface 112 (step S214).

When the correction operation has been input to the input interface 112, the answer determination function 122 corrects various types of determination results in accordance with the correction operation (step S216).

Subsequently, in a weighting coefficient decision function 123, a degree of reliability of answer data is decided on as a weighting coefficient on the basis of a result of determining whether or not the consistency in the answer data is medically valid (step S218).

Subsequently, in an estimation function 124, answer data (i.e., weighted answer data) for which the weighting coefficient is decided on in the weighting coefficient decision function 123 is input with respect to the trained model MDL defined by the model information MI stored in the memory 141 (step S220).

Subsequently, in the estimation function 124, a disease estimation result is acquired from a trained model MDL to which at least the weighted answer data of the medical diagnosis target person has been input (step S222).

Subsequently, in the output control function 125, a result of estimating the trained model MDL is output via an output interface 113 (step S224). Thereby, the process of the present flowchart ends.

FIG. 18 is a diagram showing an example of a display screen of a result of determining consistency and validity in an answer. In an example shown in FIG. 18, it is determined that second and third answer data is inconsistent with first answer data (answer data at the top of FIG. 18), but second and third answer data is (ii) answer data in which there is inconsistency (there is a change) but the inconsistency is medically valid. In such a case, the output control function 125 may cause the display 113a to display a button B1 for correcting the determination result and a button B2 for canceling the determination result without correcting the determination result. For example, when the medical staff member selects the button B1 using an input interface 112, a determination result indicating that the second and third answer data is (iii) answer data in which there is inconsistency (there is a change) and the inconsistency is medically invalid may be corrected in the answer determination function 122.

According to the third embodiment described above, the processing circuitry 120 outputs results of determining the consistency and validity in the answer via the output interface 113. When a correction operation is input to the input interface 112, the processing circuitry 120 corrects the determination results according to the correction operation. Thereby, the weighting coefficient of answer data can be determined more appropriately, such that the accuracy of disease estimation based on the trained model MDL can be further improved.

### OTHER EMBODIMENTS

Hereinafter, other embodiments will be described. Although the user interface 10 and the medical information processing device 100 have been described as devices different from each other in the above-described embodiment, the present invention is not limited thereto. For example, the user interface 10 and the medical information processing device 100 may be integrated into one device. For example, the processing circuitry 20 of the user interface 10 may further include the answer determination function 122, the weighting coefficient decision function 123, and the estimation function 124 provided in the processing circuitry 120 of the medical information processing device 100 in addition to the acquisition function 21, the output control function 22, and the communication control function 23. In this case, the user interface 10 can perform the above-described processes of various types of flowcharts standalone (offline).

According to at least one embodiment described above, the processing circuitry 120 of the medical information processing device 100 acquires answer data (an example of “a first answer”) of a medical diagnosis target person to a medical interview question made at a first timing and answer data (an example of “a second answer”) of the medical diagnosis target person to the medical interview question made at a second timing different from the first timing and determines whether or not the answer data is consistent. Thereby, it is possible to reduce an influence of a change in the answer when medical treatment is performed on the patient on the basis of the answer of the patient to the medical interview question.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

In relation to the above embodiments, the following appendixes are disclosed as aspects and selective features of the invention.

(Appendix 1)

A medical information processing system includes:

an acquirer configured to acquire a first answer of a target person to a medical interview question made at a first timing and a second answer of the target person to a medical interview question made at a second timing different from the first timing; and an answer determiner configured to determine whether or not the first answer and the second answer are consistent.

(Appendix 2)

Content of the medical interview question of the first timing may be the same as content of the medical interview question of the second timing and an expression of the medical interview question of the first timing may be different from an expression of the medical interview question of the second timing.

(Appendix 3)

The acquirer may further acquire state data indicating at least one of a state of the target person at the first timing and a state of the target person at the second timing. The answer determiner may further determine whether or not the first answer or the second answer is consistent with the state data.

(Appendix 4)

The state data may be image data or sound data of the target person.

(Appendix 5)

The answer determiner may determine whether or not inconsistency in the first answer and the second answer is medically valid when the first answer and the second answer are inconsistent.

(Appendix 6)

The answer determiner may determine whether or not the inconsistency in the first answer and the second answer is medically valid on the basis of a time interval between the first timing and the second timing.

(Appendix 7)

The answer determiner may determine whether or not the inconsistency is medically valid on the basis of a position of the target person at each of the first timing and the second timing.

(Appendix 8)

The medical information processing system may further include a user interface capable of being operated by the target person. The answer determiner may determine whether or not the inconsistency is medically valid on the basis of an operation quantity on the user interface when the target person has input the first answer to the user interface and an operation quantity on the user interface when the target person has input the second answer to the user interface.

(Appendix 9)

The user interface may be a touch interface. The answer determiner may determine whether or not the inconsistency is medically valid on the basis of at least one of a pressure, time, and the number of operations when the target person has input the first answer to the touch interface by touch and at least one of a pressure, time, and the number of operations when the target person has input the second answer to the touch interface by touch.

(Appendix 10)

The user interface may be a voice user interface. The answer determiner may determine whether or not the inconsistency is medically valid on the basis of at least one of a sound pressure, time, and the number of operations when the target person has input the first answer to the voice user interface by voice and at least one of a sound pressure, time, and the number of operations when the target person has input the second answer to the voice user interface by voice.

(Appendix 11)

The medical information processing system may further include a weighting coefficient decider configured to decide on degrees of reliability of the first answer and the second answer as weighting coefficients on the basis of a result of determining whether or not the inconsistency is medically valid.

(Appendix 12)

The weighting coefficient decider may relatively increase the weighting coefficient in a case where the inconsistency is medically valid as compared with a case where the inconsistency is medically invalid.

(Appendix 13)

The weighting coefficient decider may further decide on the weighting coefficient in accordance with a type of disease from which the target person may suffer.

(Appendix 14)

The weighting coefficient decider may relatively decrease the weighting coefficient in a case where the inconsistency is medically valid as compared with a case where the inconsistency is medically invalid when the disease is a mental disease.

(Appendix 15)

The medical information processing system may further include an estimator configured to estimate a disease of the target person on the basis of at least one of the first answer and the second answer for which the weighting coefficient has been decided on.

(Appendix 16)

The estimator may input at least one of the first answer and the second answer for which the weighting coefficient has been decided on with respect to a trained model and estimate the disease of the target person on the basis of information output according to the trained model. The trained model may be a model subjected to supervised learning on the basis of training data in which a disease from which the patient suffers is associated with an answer to a medical interview question from the patient as a correct label.

(Appendix 17)

The medical information processing system may further include a display controller configured to cause a display to display the first answer and the second answer in chronological order.

(Appendix 18)

The display controller may change a display mode of the first answer and the second answer when the first answer and the second answer are inconsistent.

(Appendix 19)

A medical information processing method includes

acquiring, by a computer a first answer of a target person to a medical interview question made at a first timing and a second answer of the target person to a medical interview question made at a second timing different from the first timing; and

determining, by the computer, whether or not the first answer and the second answer are consistent.

(Appendix 20)

A program for causing a computer to execute:

a function of acquiring a first answer of a target person to a medical interview question made at a first timing and a second answer of the target person to a medical interview question made at a second timing different from the first timing; and

a function of determining whether or not the first answer and the second answer are consistent.

What is claimed is:

1. A medical information processing system comprising processing circuitry configured to:
acquire a first answer of a target person to a medical interview question made at a first timing and a second answer of the target person to a medical interview question made at a second timing different from the first timing; and
determine whether or not the first answer and the second answer are consistent.

2. The medical information processing system according to claim 1,
wherein content of the medical interview question of the first timing is the same as content of the medical interview question of the second timing, and
wherein an expression of the medical interview question of the first timing is different from an expression of the medical interview question of the second timing.

3. The medical information processing system according to claim 1, wherein the processing circuitry further acquires state data indicating at least one of a state of the target person at the first timing and a state of the target person at the second timing and determines whether or not the first answer or the second answer is consistent with the state data.

4. The medical information processing system according to claim 3, wherein the state data is image data or sound data of the target person.

5. The medical information processing system according to claim 1, wherein the processing circuitry determines whether or not inconsistency in the first answer and the second answer is medically valid when the first answer and the second answer are inconsistent.

6. The medical information processing system according to claim 5, wherein the processing circuitry determines whether or not the inconsistency in the first answer and the second answer is medically valid on the basis of a time interval between the first timing and the second timing.

7. The medical information processing system according to claim 5, wherein the processing circuitry determines whether or not the inconsistency is medically valid on the basis of a position of the target person at each of the first timing and the second timing.

8. The medical information processing system according to claim 5, further comprising a user interface capable of being operated by the target person,
wherein the processing circuitry determines whether or not the inconsistency is medically valid on the basis of an operation quantity on the user interface when the target person has input the first answer to the user interface and an operation quantity on the user interface when the target person has input the second answer to the user interface.

9. The medical information processing system according to claim 8,
wherein the user interface is a touch interface, and
wherein the processing circuitry determines whether or not the inconsistency is medically valid on the basis of at least one of a pressure, time, and the number of operations when the target person has input the first answer to the touch interface by touch and at least one of a pressure, time, and the number of operations when the target person has input the second answer to the touch interface by touch.

10. The medical information processing system according to claim 8,
wherein the user interface is a voice user interface, and
wherein the processing circuitry determines whether or not the inconsistency is medically valid on the basis of at least one of a sound pressure, time, and the number of operations when the target person has input the first answer to the voice user interface by voice and at least one of a sound pressure, time, and the number of operations when the target person has input the second answer to the voice user interface by voice.

11. The medical information processing system according to claim 5,
wherein the processing circuitry further decides on degrees of reliability of the first answer and the second answer as weighting coefficients on the basis of a result of determining whether or not the inconsistency is medically valid.

12. The medical information processing system according to claim 11, wherein the processing circuitry relatively increases the weighting coefficient in a case where the inconsistency is medically valid as compared with a case where the inconsistency is medically invalid.

13. The medical information processing system according to claim 11, wherein the processing circuitry further decides on the weighting coefficient in accordance with a type of disease from which the target person may suffer.

14. The medical information processing system according to claim 13, wherein the processing circuitry relatively decreases the weighting coefficient in a case where the inconsistency is medically valid as compared with a case where the inconsistency is medically invalid when the disease is a mental disease.

15. The medical information processing system according to claim 11, wherein the processing circuitry further estimates a disease of the target person on the basis of at least one of the first answer and the second answer for which the weighting coefficient has been decided on.

16. The medical information processing system according to claim 15,
wherein the processing circuitry inputs at least one of the first answer and the second answer for which the weighting coefficient has been decided on with respect to a trained model and estimates the disease of the target person on the basis of information output according to the trained model in accordance with a process in which at least one of the first answer and the second answer has been input, and
wherein the trained model is a model subjected to supervised learning on the basis of training data in which a disease from which the patient suffers is associated with an answer to a medical interview question from the patient as a correct label.

17. The medical information processing system according to claim 1, wherein the processing circuitry further causes a display to display the first answer and the second answer in chronological order.

18. The medical information processing system according to claim 17, wherein the processing circuitry changes a display mode of the first answer and the second answer when the first answer and the second answer are inconsistent.

19. A medical information processing method using a computer, the medical information processing method comprising:
acquiring a first answer of a target person to a medical interview question made at a first timing and a second answer of the target person to a medical interview question made at a second timing different from the first timing; and
determining whether or not the first answer and the second answer are consistent.

20. A non-transitory computer-readable storage medium storing a program for causing a computer to execute:
acquiring a first answer of a target person to a medical interview question made at a first timing and a second answer of the target person to a medical interview question made at a second timing different from the first timing; and
determining whether or not the first answer and the second answer are consistent.
